(19) United States
(12) Patent Application Publication
(10) Pub. No.: US 2024/0416115 A1
(43) Pub. Date: Dec. 19, 2024

(54) EPICARDIAL PACING SYSTEM WITH BIODEGRADABLE LEADS

(71) Applicant: Pirooz S. Mofrad, Bethesda, MD (US)

(72) Inventor: Pirooz S. Mofrad, Bethesda, MD (US)

(21) Appl. No.: 18/335,763

(22) Filed: Jun. 15, 2023

(51) Int. Cl. A61N 1/05

(52) U.S. Cl. CPC A61N 1/0595; A61N 1/059

(57) ABSTRACT

An improved system for temporary cardiac pacing addresses the problem associated with heart tissue damage caused by removal of the temporary pacing leads. In the improved system the leads biodegrade and thus need not be removed. This, in turn, allows the surgeon to attach the leads more firmly to the heart.

Described here is an improvement on devices for temporary cardiac pacing. Specifically, the invention eliminates the risky step of removal of the pacing wires, or leads.

### Heart Block

For a short time following thoracic or cardiothoracic surgery, the heart can fail to resume beating normally and instead “asystole” or “heart block” occurs. In lay terms, the heart does not properly “wake up.” This occurs as high as 40 percent according to some studies. If the condition is left untreated, stroke or heart failure can result.

In order to resolve this condition, temporary postoperative pacing is employed. In a preferred embodiment, this treatment can be used prophylactically where heart block is considered likely. Under temporary pacing a heart is supported in the recovery time frame after surgery. During this time, should the heart pause or the heart beat slow (bradycardia) the temporary pacing can take over until the heart fully “wakes up” and is able to return to normal operation.

Temporary postoperative pacing should not be confused with permanent pacing, a quite different procedure. The pulse generator for a permanent pacemaker is implanted inside the patient's chest. The pulse generator for temporary pacing, in contrast, remains outside the body, commonly on a bedside table, and only the pacing leads enter the patient's body. As described in more detail below, the pacing leads are typically left in place until the end of the patients recovery period, approximately 4 days. The leads are then removed. It should be noted that on rare occasions the heart fails to return to normal operation and in those cases, and those cases alone, a permanent pacemaker can be implanted.

Importantly, despite the dangers, temporary cardiac pacing should not be universally applied to all heart patients after their surgery. The risks associated with setting up temporary postoperative pacing may outweigh the benefits of insuring against atrial fibrillation. Patients without significant preconditions are at low risk of atrial fibrillation and temporary pacing will not be necessary. These patients are simply monitored post operatively and further treatment is only called for in those relatively rare cases where atrial fibrillation develops. For others heart patients, such as those with a third degree heart block (e.g. no apparent conduction between the P waves and QRS complexes) temporary pacing is universally considered necessary. For these patients, a temporary pacemaker will be implanted immediately after surgery.

In still other conditions it is difficult to determine beforehand whether a patients will benefit from temporary pacing. In these cases, it is difficult to decide whether to use temporary postoperative pacing or not. The surgeon must carefully weigh the risks with the benefits of temporary postoperative pacing.

### Temporary Pacing Traditional Setup

After the cardiac surgery is completed, but before the patient's chest is sewn up, the pacing wires are attached. One end is attached directly to the heart muscle, or epicardium. The other end exits through the chest and is connected to the external pulse generator.

The pacing lead, or wire, like most wires, is made up of a core of conducting material surrounded by insulation. At the ends of the wire, the conducting material is exposed, and this portion of the lead is placed in direct contact with the outside surface of the heart.

There are two common pacing wire systems, unipolar and bipolar. In a unipolar system a single wire (the negative lead, or anode) is attached to the epicardium. The positive lead is attached at a distance in the subcutaneous tissues.

In a bipolar system the two wires are combined into a single lead. The conductive core is exposed at the ends, like each of the unipolar leads. But in the case of a bipolar system, the ends are both placed in contact with the epicardial surface, typically about 8 mm apart. The negative lead, or anode, is usually placed distal to the positive lead. The other ends of the leads are connected to the bedside pulse generator similar to the unipolar system.

The bipolar electrodes are more suitable for use in dual chamber applications. The pacing current travels a shorter distance, relative to unipolar systems, and thus the electrical potential required to bring the myocardium to threshold is less than the unipolar system. This lower potential means there is less risk that a lead implanted in one chamber will interfere with the operation of a lead planted in the other chamber.

### Attachment of the Lead to the Heart

A major risk of temporary postoperative pacing is associated with removing the pacing wire, routinely place on the surface of the heard after surgery. The pacing wire can tear the heart tissue leading to internal bleeding and loss of blood pressure. The patient must be immediately treated to repair the damage and stop any significant, persistent bleeding. Unfortunately, it is often difficult to immediately diagnose a tear without reopening the patient's chest to expose the affected area.

To properly attach the temporary pacing leads, the surgeon must find an elusive balance. The attachment cannot be too secure, or one risks tearing heart tissue when the lead is removed. But the attachment must be secure enough so that the leads do not detach by normal movement during the patients post-operative pacing period.

As a result, doctors use several methods of attachment. Typically, a slight curve is added to the tip of the pacing wires which secures the lead to the heart. For additional security, the lead can be loosely sutured in place, although some surgeons prefer no suturing.

To reduce the incidence of tearing surgeons are left to monitor the pressure needed to pull the leads at the end of the pacing period. If the tension is to high, the surgeon will often cut the wire at the skin. The wire will then retract into the chest. With luck the leads will remain in the patients test without causing further problems.

Still other surgeons never pull out the lead. They always use the cut and retract method. Other surgeons chose to be more conservative about which patients require prophylactic post-operative pacing.

The post-operative patient must also be conscious of the danger of premature lead detachment and limit their movements during the post-operative pacing period.

### Biodegradable Pacemaker

The traditional post-operative pacing systems have several disadvantages. Bacterial can be present on the pacing leads and could cause infections if carried into the body when the pacing leads are attached to the heart. Also, the removal of the pacing leads can damage the myocardium causing tears or perforations. To avoid these problems, an alternative proposed is a leadless, battery-free, fully implantable cardiac pacemaker that, after the post-operative pacing was completed, would completely dissolve inside the patents body.

There are several problems with this proposal. First, the pulse generation component, with design alterations making it both miniature and fully biodegradable, will be less reliable than the traditional, external counterpart. Also, the components left inside the body, while technically “biodegradable” can still cause problems. The device can be dislodged and cause tissue damage prior to full dissolution. Also, certain patients can have an adverse reaction to the substances produced and spread through the body as the device dissolves.

The device of this invention is an improved pacing wire. It does not need to be removed from the patient, but dissolves harmlessly, safely after the completion of the postoperative of temporary pacing.

### BRIEF DESCRIPTION OF THE DRAWINGS

FIG. 1A illustrates implantation of a bipolar version of the device.

FIG. 1B illustrates implantation of a unipolar version of the device.

FIG. 2 illustrates the assembly used to implant the electrode.

FIGS. 3A-E illustrate a preferred method of attaching the electrode to the epicardium.

### DETAILED DESCRIPTION OF PREFERRED EMBODIMENTS

Unlike standard leads for temporary cardiac pacing, the leads of the invention are made of materials that the human body can dissolve. This is analogous to absorbable sutures commonly used to treat internal wounds with the following differences. The absorbable sutures are made up of a combination of a core of absorbable conductive material surrounded by an absorbable insulator.

The inventor believes any combination of conductor and insulator will function in the disclosed system, so long at the materials will dissolve in a reasonable time after temporary pacing is completed. Preferred conductive materials include: Preferred insulating materials include various biodegradable plastics, paper products, bioresorbable silk.

Because the pacing leads do not need to be removed, the surgeon can confidently attach the pacing leads more securely to the heart. This significantly reduces the skill level necessary for this portion of the operation, allows the patient to move in the post-operative pacing period with significantly less fear of detaching the pacing leads. Also, surgeons can be more aggressive and widen the scope of patient categories who receive post-operative pacing. With the danger of tearing reduced it becomes appropriate to proscribe post-operative pacing to patents less at risk for fibrillation. All of this leads to more successful outcomes.

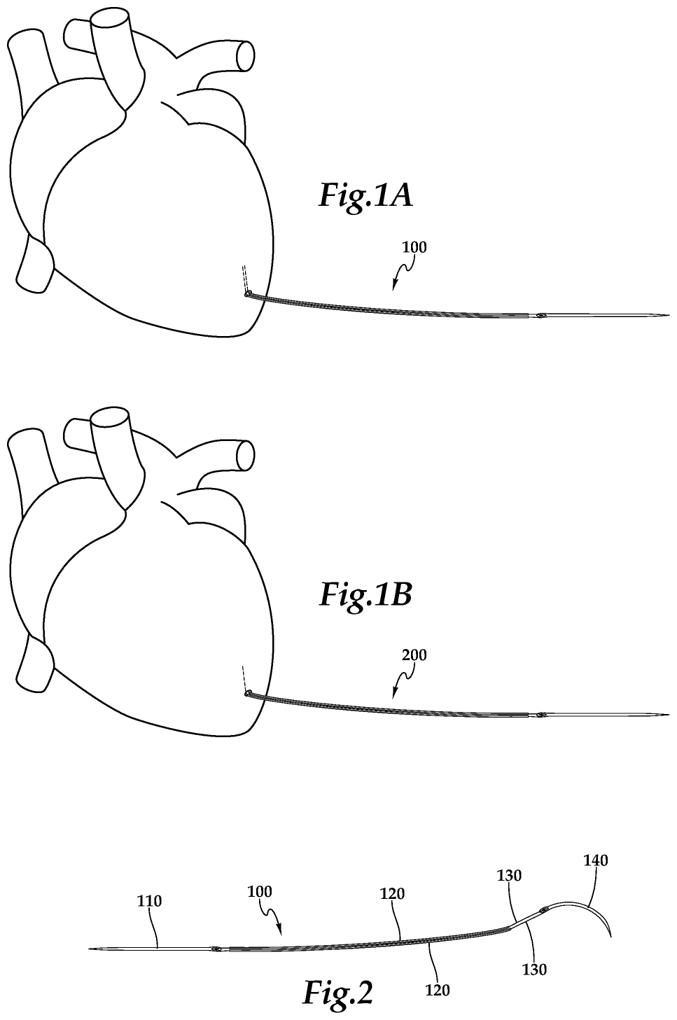

FIG. 1A illustrates a heart being implanted with bipolar degradable electrodes. The bipolar electrodes are implanted into the epicardial muscle in a process described below. The electrodes are connected to biodegradable leads which connect the electrodes to the external pulse generator. These leads are also made out of biodegradable material. In this case, a biodegradable conductor is surrounded by biodegradable insulator materials. Once implanted the device is designed to operate precisely as prior are bipolar electrodes.

Similarly, FIG. 1B illustrates the use of a unipolar electrode. The biodegradable electrodes are implanted as shown and are connected by biodegradable leads to the external pulse generator. Otherwise the device operates similarly to the prior art, non-degradable version.

FIG. 2 shows the assembly of components 100 used to implant the electrode as shown in FIG. 1A. In a typical use of the device, a patient has just undergone an open heart surgical procedure in which the chest cavity has been opened. The needle 140 is used to imbed electrodes 130 into the exposed heart, specifically into the epicardial muscle. See below. A second needle 110 is then used to pierce the chest wall so that the leads exit the chest cavity in a region of the chest adjacent to the site of electrode implantation.

After the chest cavity is closed, the patient will be, typically, transferred to an intensive care unit where the leads exiting the patient's chest will be connected to the external pulse generator. At the end of the recovery period the pulse generator is disconnected. The leads may be trimmed back at the chest wall. The portion of the device inside the chest, the electrodes and leads, will not be removed from the patient. They will dissolve inside the chest cavity over a period of weeks or months.

In prior art procedures electrode implantation must be performed with precision. If the electrode is not implanted sufficiently firm, the electrode can work loose prematurely. On the other hand, if the electrode in implanted too firmly, when the electrode is removed after the recovery period it can tear and damage the heart. Because either extreme can lead to complications, electrode implantation can require great skill and extensive training.

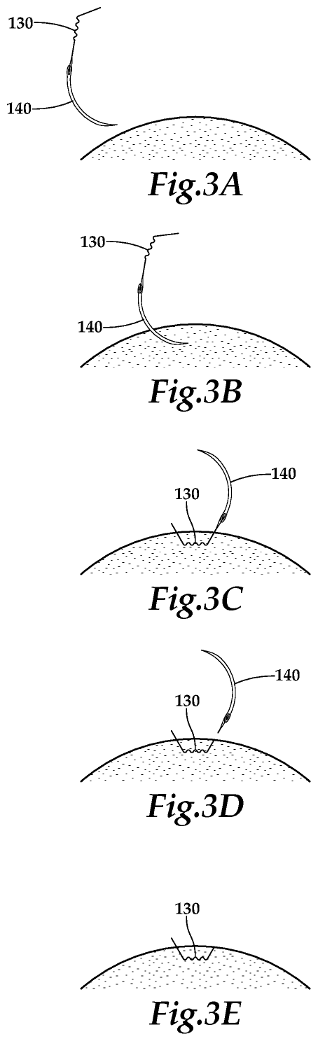

The electrode disclosed here can be implanted into the chest as shown in FIGS. 3A-E. Because the electrode will eventually dissolve the danger from tearing present in the prior art device is nonexistent. Accordingly, the electrodes can be implanted much more firmly into the epicardial muscle to guard against the only danger, premature loosening and separation of the electrode from the heart.

The first step in implanting the electrode is to pierce the heart muscle with the needle as shown in FIGS. 3A-B. The needle is curved in one embodiment, illustrated here, but can be any shape the surgeon choses. The needle draws the electrode into the heart tissue and the needle then directed out of the tissue, leaving the electrode embedded, as shown in FIG. 3C. The needle is then cut off and separated from the electrode as shown in FIG. 3D. FIG. 3E illustrates the embedded electrode.

While a specific use of the biodegradable electrodes and biodegradable leads are described, any other use is contemplated in which an electrode is to be implanted in a patients tissue and, afterword, the removal of the electrode is undesirable.

What is claimed is:

1. An epicardial pacing wire assembly, that does not need to be removed from a patient, for use with a pacemaker for temporary pacing after cardiac surgery,
said pacing wire assembly comprising;
an epicardium needle, an electrode segment, an insulated segment and a chest wall piercing needle,
the epicardium needle comprises a tip configured to be secured to a patient's epicardium and a base attached to the electrode segment,
the electrode segment comprising one or more linear wire electrodes configured to contact the epicardium and deliver electric energy that stimulates myocardium action potential, one end of the electrode segment configured to attach to the epicardium needle and the other end configured to attach to the insulated segment,
the insulated segment comprising,
one end of the insulating segment connected to the electrode segment and the other end connected to the chest wall piercing needle,
wires running from each of the one or more electrodes at one end of the segment through the central longitudinal axis and out the other end of the segment,
insulating composition surrounding the wires and insulating them from the surrounding environment,
the chest wall piercing needle comprising one end connected to the insulated segment and a free end configured to pierce the patient's chest wall so that a surgeon can pierce the chest well from inside though to the outside and drawn the connected elements of the pacing wire assembly outside the patient's chest,
wherein the electrode segment and insulated segment are made of biodegradable material and need not be removed following the temporary pacing.

2. The epicardial pacing wire assembly as defined in claim 1 wherein said electrodes and wires further comprise melanin.

3. The epicardial pacing wire assembly as defined in claim 1 wherein said insulating composition is selected from the group of silk or cellulose.

4. The epicardial pacing wire assembly as defined in claim 1 wherein each of said electrode and said wire are a seamless one piece length of the same material.

5. The epicardial pacing wire assembly as defined in claim 1 wherein said electrode segment further comprises a single electrode for unipolar epicardial pacing.

6. The epicardial pacing wire assembly as defined in claim 1 wherein said electrode segment further comprises two electrodes for bipolar epicardial pacing.
